(19) United States
(12) Patent Application Publication
(10) Pub. No.: US 2026/0232714 A1
(43) Pub. Date: Aug. 13, 2026

(54) COMPOSITION CONTAINING NICOTINAMIDE MONONUCLEOTIDE

(71) Applicant: MEIJI HOLDINGS CO., LTD., Tokyo (JP)

(72) Inventors: Jun WAKABAYASHI, Tokyo (JP); Masashi MORIFUJI, Tokyo (JP); Seiichiro HIGASHI, Tokyo (JP); Aiko MANJI, Tokyo (JP)

(73) Assignee: MEIJI HOLDINGS CO., LTD., Tokyo (JP)

(21) Appl. No.: 19/154,173

(22) PCT Filed: Feb. 6, 2024
(86) PCT No.: PCT/JP2024/003788

§ 371 Date: Aug. 6, 2025

(30) Foreign Application Priority Data
Feb. 7, 2023 (JP) 2023-016893

(51) Int. Cl. A61K 31/706; A61K 9/00

(52) U.S. Cl. CPC A61K 31/706; A61K 9/006

(57) ABSTRACT

There is provided a composition that can rapidly transfer a large amount of nicotinamide mononucleotide into the blood after ingestion and thereby increase the concentration of nicotinamide mononucleotide in the blood. A composition comprising nicotinamide mononucleotide is provided, which is for increasing the concentration of nicotinamide mononucleotide in human blood and in a form suitable for mucosal application.

### TECHNICAL FIELD

The present invention relates to a composition comprising nicotinamide mononucleotide. The present invention is useful in the fields of health and nutritional metabolism, as well as manufacture of foods and beverages for them, and so forth.

### BACKGROUND ART

Nicotinamide mononucleotide (NMN) is a precursor of nicotinamide adenine dinucleotide (NAD), which is necessary for humans to maintain vital functions, and is a compound that belongs to vitamin B3. NMN has been reported so far to have many beneficial effects, including anti-aging effects, when administered to humans and mice (Non-patent document 1). While the amount of NAD in the body declines in old age, administration of NMN, a precursor of NAD, is thought to be effective to deal with this problem, and supplementation of NMN is being examined with expecting anti-aging effect thereof.

However, it has been reported that if NMN is administered orally, most of it is degraded in the intestinal tract through metabolism by intestinal bacteria (Non-patent document 2). Even when absorbed from the intestinal tract, most of it becomes nicotinamide (NAM) due to the first-pass effect in the liver (Non-patent document 3). Therefore, in most studies with mice, NMN was administered intraperitoneally, and studies using oral administration have been rarely reported.

By the way, the sublingual absorption route exists as an absorption route for pharmaceuticals and so forth, and it is known that sublingual absorption allows direct absorption of substances into the blood. Patent documents 1 and 2 describe solid dosage forms for transmucosal drug delivery using special water-soluble polymers containing active pharmaceutical ingredients (APIs), and NMN is exemplified as one example of API.

### PRIOR ART REFERENCES

### Patent Documents

Patent document 1: International Publication WO2021/231860
Patent document 2: US2023/0121000

### Non-Patent Documents

Non-patent document 1: Hong W et al, Front Cell Dev Biol. 2020 Apr. 28; 8:246.
Non-patent document 2: Kim L et al, bioRxiv; 2020
Non-patent document 3: Liu L et al, Cell Metab. 2018 May 1; 27 (5): 1067-1080

### SUMMARY OF THE INVENTION

### Problem to be Solved by the Invention

The kinetics of NMN in humans when orally administered is not clear. According to the recent study of the inventors of the present invention, it has been found that the blood concentration of NMN does not increase within one hour after oral administration of NMN to humans.

In order for NMN to exert the useful effects thereof, it is considered important to rapidly transfer a large amount of NMN into the blood and increase the blood NMN concentration.

### Means for Solving the Problem

Sublingual administration of NMN may result in rapid transfer of NMN into the blood and allow delivery of more NMN to the peripheral tissues through the body circulation. Therefore, the inventors of the present invention experimentally verified whether sublingual administration of NMN increases the blood concentration of NMN compared with oral administration, and accomplished the present invention.

The present invention provides the followings.
[1] A composition for increasing concentration of NMN in human blood, wherein the composition comprises nicotinamide mononucleotide (NMN) and is in a form suitable for mucosal application.
[2] The composition according to 1, which is an agent for application to an oral mucosa.
[3] The composition according to 1 or 2, which is for administration or ingestion of 200 mg or more per day of NMN.
[4] The composition according to any one of 1 to 3, which is for achieving a maximum blood concentration of 0.20 μg/mL or higher within 1 hour after administration or ingestion.
[5] The composition according to any one of 1 to 4, which is for administration to or ingestion by elderly persons.
[6] The composition according to any one of 1 to 5, which is for anti-aging.
[7] The composition according to any one of 1 to 6, which is for improving the oral environment.
[8] The composition according to any one of 1 to 7, which is a food composition or a pharmaceutical composition.
[9] A composition for use in a method for increasing concentration of NMN in human blood, wherein the composition comprises NMN and is in a form suitable for mucosal application. A use of NMN in manufacture of a composition in a form suitable for mucosal application for increasing concentration of NMN in human blood. A method or a non-therapeutic method for increasing concentration of NMN in human blood, which comprises a step of administering a composition comprising NMN in a form suitable for mucosal application. A use or a non-therapeutic use of a composition comprising NMN in a form suitable for mucosal application for increasing concentration of NMN in human blood.
[10] The composition, use in manufacture, method or non-therapeutic method, or use or non-therapeutic use according to 9, wherein the composition is an agent for application to an oral mucosa.
[11] The composition, use in manufacture, method or non-therapeutic method, or use or non-therapeutic use according to 9 or 10, wherein the composition is for administration or ingestion of 200 mg or more per day of NMN.
[12] The composition, use in manufacture, method or non-therapeutic method, or use or non-therapeutic use according to any one of 9 to 11, wherein the composition is for achieving a maximum blood concentration of 0.20 μg/mL or higher within 1 hour after administration or ingestion of the composition.
[13] The composition, use in manufacture, method or non-therapeutic method, or use or non-therapeutic use according to any one of 9 to 12, wherein the composition is for administration to or ingestion by elderly persons.
[14] The composition, use in manufacture, method or non-therapeutic method, or use or non-therapeutic use according to any one of 9 to 13, wherein the composition is for anti-aging.
[15] The composition, use in manufacture, method or non-therapeutic method, or use or non-therapeutic use according to any one of 9 to 14, wherein the composition is for improvement of the oral environment.
[16] The composition, use in manufacture, method or non-therapeutic method, or use or non-therapeutic use according to any one of 9 to 15, wherein the composition is a food composition or a pharmaceutical composition.

The present invention provides the followings.
[1] A composition for increasing concentration of NMN in human blood, wherein the composition comprises nicotinamide mononucleotide (NMN) and is made into a preparation for mucosal application.
[2] The composition according to 1, which is an agent for application to an oral mucosa.
[3] The compositions according to 1 or 2, which is for administration of 200 mg or more per day of NMN.
[4] The composition according to any one of 1 to 3, which is for achieving a maximum blood concentration of 0.20 μg/mL or higher within 1 hour after administration.
[5] The composition according to any one of 1 to 4, which is for administration to elderly persons.
[6] The composition according to any one of 1 to 5, which is for anti-aging.
[7] The composition according to any one of 1 to 6, which is for improving the oral environment.

### Effects of the Invention

According to the present invention, NMN concentrations in human blood can be increased.

NMN used as the active ingredient of the composition of the present invention has been ingested from old days, and the composition of the present invention can safely attain the desired effects.

### BRIEF DESCRIPTION OF THE DRAWINGS

FIG. 1 Ratios of change in blood NMN concentration observed in Test 1, wherein the values are relative values based on the value obtained before the NMN administration (0 minute), which is taken as 100.

FIG. 2 Ratios of change in blood NMN concentration observed in Test 2, wherein the values are relative values based on the value obtained before the NMN administration (0 minute), which is taken as 100. (A) Ratios of change in NMN concentration observed after oral or sublingual ingestion by the test subject A, and (B) Ratios of change in NMN concentration observed after oral or sublingual ingestion by the test subject B.

### MODES FOR CARRYING OUT THE INVENTION

### [Active Ingredient]

The composition of the present embodiment comprises a nicotinamide adenine dinucleotide (NAD)-related substance or a salt thereof acceptable as food or pharmaceutical as the active ingredient.

The NAD-related substance is NAD or a substance that can be converted into NAD in vivo. NAD functions in vivo as a coenzyme for various dehydrogenases and can take two states: oxidized type (NAD⁺) and reduced type (NADH). Examples of the NAD-related substance include nicotinamide riboside (NR), NAD, nicotinamide (NAM, also referred to as niacinamide), nicotinic acid (NA), NaAD (nicotinic acid adenine dinucleotide), and NaMN (nicotinic acid mononucleotide). With respect to the present invention, NAD refers to either oxidized type (NAD⁺) or reduced type (NADH), unless otherwise stated.

The NAD-related substance may be a nicotinamide mononucleotide derivative, which is a compound represented by the general formula (I).

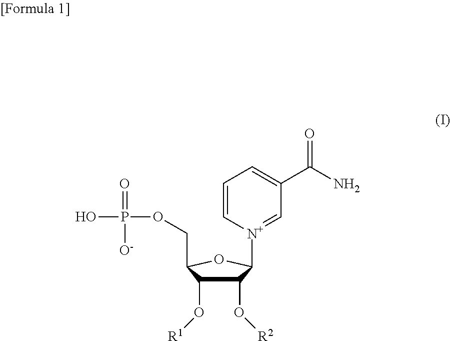

In the formula, R¹ and R² are each independently an acyl group having 6 to 16 carbons, and the hydrocarbon group attached to the carbonyl carbon of the acyl group is a linear or branched-chain saturated or unsaturated hydrocarbon group. Such a compound can be produced by an acylation reaction of NMN with any acylating agent selected from the group consisting of a carboxylic acid having an acyl group having 6 to 16 carbons and having a linear or branched-chain saturated or unsaturated hydrocarbon group attached to the carbonyl carbon, a halide of the carboxylic acid, and an anhydride of the carboxylic acid in a solvent comprising 20 mass % or more of a strongly acidic liquid having a pKa of 2.0 or lower (see WO2017/110317).

In one embodiment, the NAD-related substance may be included in the composition as a salt acceptable as food or pharmaceutical. The salt acceptable as food or pharmaceutical refers to any salt selected from the group consisting of nitrate, sulfate, carbonate, hydrogencarbonate, halide salt, formate, acetate, citrate, tartrate, oxalate, fumarate, salt of a saturated or unsaturated fatty acid having 3 to 20 carbons, salt of carnitine or a derivative thereof, salt of hydroxycitric acid or a derivative thereof, salt of ascorbic acid or a derivative thereof, salt of ascorbyl phosphate and a derivative thereof, sodium salt, potassium salt, calcium salt, magnesium salt, zinc salt, and ammonium salt.

In a preferred embodiment, the composition comprises NMN as the active ingredient.

NMN has two types of optical isomers, α-type and β-type, and NMN refers to β-type NMN (β-nicotinamide mononucleotide) with respect to the present invention, unless otherwise stated. NMN is an intermediate metabolite of NAD⁺. Hereinafter, the present invention may be explained by referring to a case where the active ingredient is NMN among NAD-related substances and salts thereof acceptable as food or pharmaceutical as an example, but those skilled in the art can understand the present invention by appropriately applying such explanations to the cases where the active ingredient is an NAD-related substance other than NMN or a salt thereof acceptable as food or pharmaceutical. The term active ingredient means an ingredient that contributes to the intended purpose, and with regard to foods with functional claims, it may be referred to as a functionally-associated ingredient.

NMN, NAD analogues, and salts thereof acceptable as food or pharmaceutical can be produced by various methods. They may be synthesized or extracted from natural products comprising the substances, or cultures of yeast or others.

### [Form]

In one embodiment, the dosage form of the composition is a form suitable for mucosal application. Being in a suitable form is also expressed as “being made into a preparation”. The term preparation means a product in a form suitable for use obtained by adding additives to the active ingredient, NMN, as required, or means the process of producing the preparation. The composition may be a food composition or a pharmaceutical composition as discussed below, and the explanations of the preparation described in this specification apply to both food and pharmaceutical compositions.

Examples of the form suitable for mucosal application include agents for oral mucosa application (also referred to as topical agents for oral mucosa application), and topical agents for application to other mucosae (e.g., suppositories and vaginal agents), and agents for oral mucosa application are preferred.

In one particularly preferred embodiment, the composition is in a form suitable for sublingual administration. Sublingual administration means placing a drug under the tongue and allowing it to be absorbed through the oral mucosa. Hereinafter, the present invention and embodiments thereof may be explained for the case of using a form suitable for sublingual administration as an example, but those skilled in the art can understand the present invention and embodiments thereof by appropriately applying such descriptions to other agents for mucosal application.

Such agents for oral mucosal application can be in a solid or liquid form, and solid form is preferred. Examples of solid agents for oral mucosa application include powders, fine powders, tablets, films, lozenge tablets, pills, strips, buccals, drops, candies, gums or gummies. Particularly preferred forms when the composition is a food include tablet confectionaries, gummies, drops, candies, gums, and troches.

### [Use]

### (Function/Action)

The composition of the present invention is used to increase the NMN concentration in the blood in humans. When it is administered sublingually, intranasally, or buccally, the active ingredient, NMN, is rapidly absorbed from the mucosa, and can reach the systemic bloodstream without being subjected to the first-pass effect in the liver.

The blood kinetics of NMN administered orally to humans are revealed in this specification. According to the study of the inventors of the present invention, the blood NMN concentration after oral administration is almost unchanged at all the other blood collection points compared with before the ingestion of NMN (0 minute). It is considered that this is because orally administered NMN is metabolized by intestinal bacteria and the liver.

On the other hand, the blood NMN concentration observed after sublingual administration of NMN can increase up to about 15-fold compared with that observed before the ingestion of NMN. More specifically, when a certain amount of NMN is sublingually administered, the maximum blood concentration can be reached within 1 hour, preferably within 45 minutes, more preferably within 35 minutes, more preferably within 25 minutes, more preferably within 15 minutes, more preferably within 10 minutes, further preferably within 7 minutes, after the administration. In some test subjects, the maximum blood concentration can be reached very quickly, e.g., after about 5 minutes from the administration.

When a certain amount of NMN is sublingually administered, the maximum blood concentration can increase several-fold, e.g., 3-fold or more, preferably 5-fold or more, more preferably 7-fold or more, more preferably 10-fold or more, even more preferably 12-fold or more, compared with that observed before the ingestion of NMN. In another aspect, sublingual administration of a certain amount of NMN provides a maximum blood concentration of, for example, 0.2 μg/mL or higher, preferably 0.4 μg/mL or higher, more preferably 0.8 μg/mL or higher, more preferably 2 μg/mL or higher, more preferably 3 μg/mL or higher, even more preferably 4 μg/mL or higher. In some test subjects, the maximum blood concentration can reach 5 μg/mL or higher.

In one embodiment, the composition is used for the treatment of a disease or condition that is ameliorated by increasing blood concentration of NMN.

In one embodiment, it is considered that the composition can allow a tissue in which Slc12a8, a NMN transporter, is highly expressed to specifically take up NMN, and thereby increase NAD in vivo in an NMN-dependent manner.

In one embodiment, the composition is used for anti-aging. The term anti-aging refers to suppressing advance of aging. NMN is a precursor of NAD, and it is known that the amount of NAD in the body decreases in old age.

In one embodiment, the composition can be used to improve the oral environment (International Publication WO2023/282283). Improvement of the oral environment includes maintaining the oral environment, preventing deterioration of the oral environment or reducing the risk of the same (prophylactic effect), and improving poor oral environment (therapeutic effect). Improvement of the oral environment is, in other words, oral care. Improvement of the oral environment may also be at least one of increase in saliva secretion and reduction in the number of bacteria in the oral cavity.

In one embodiment, the composition may be used for the treatment of at least one of xerostomia (dry mouth) and halitosis. There are various causes of xerostomia, including mouth breathing, lack of moisture, dehydration, aging, age-related decline, age-related medications, stress, disease, side effects of medication, and a combination of these factors, and the composition of the present invention may be used in any case. Examples of diseases that can cause xerostomia include diabetes, thyroid dysfunction, diabetes insipidus, and Sjogren's syndrome. Examples of drugs that may be associated with the side effects of xerostomia include antidepressants, anxiolytics, antihypertensive drugs, and analgesics. The composition of the present invention may also be used in cases where the salivary glands are exposed to radiation during cancer treatment, which may cause damage to salivary gland tissue and predispose to xerostomia.

In one embodiment, the composition may also be used for the treatment of any selected from the group consisting of oral mucositis, gingivitis, periodontitis, oral infection, oral inflammation, and halitosis.

For the present invention, treatment of a disease or condition includes reducing the risk of developing the disease, delaying onset of the disease, preventing and curing the disease, and stopping and delaying the progression of the disease. The treatment includes medical treatment performed by a physician for the purpose of treating a disease, and non-medical treatment performed by a person other than a physician, such as nutritionist, dietitian, public health nurse, midwife, nurse, clinical laboratory technician, food manufacturer, food seller, etc. The treatment also includes ingestion of specific foods or recommending ingestion of the same, dietary guidance, health guidance, nutritional guidance (including guidance on nutrition necessary for medical treatment for injured and sick persons and nutritional guidance for maintenance and promotion of good health), management of food service, and guidance necessary for nutritional improvement related to food service. For the present invention, the subject of the treatment includes humans (individuals) and non-human mammalian animals (such as companion animals). For the present invention, the term “administration (administer)” may be used not only for pharmaceutical compositions but also for food compositions. The term “administration (administer) to” may be read as “ingestion by (to make ingest)”.

### (Subject)

In one embodiment, the composition is used for administration to subjects for whom it is desirable or necessary to increase blood concentrations of NMN. Such subjects include middle-aged (40 years old or older, but younger than 65 years old), elderly (65 years old or older), persons lacking exercise, athletes, adults (15 years old or older), infants (younger than 1 year old, including newborns up to 1 month old), young children (1 year old or older, but younger than 6 years old), children (6 to 15 years old), pregnant women, maternity women, persons in or after sickness, men, and women.

In one embodiment, the composition is used for administration to middle-aged and elderly persons, preferably to the elderly. It is believed that increasing blood concentrations of NAD, which is known to decrease in the elderly, can be achieved by administering a precursor of NAD⁺ (such as nicotinamide), even if not NMN. However, in old age, nicotinamide phosphoribosyltransferase (NAMPT), the enzyme that synthesizes NMN from nicotinamide, decreases, and the synthesis of NAD⁺ (NMN) from nicotinamide is decreased. That is, the elderly cannot efficiently synthesize NAD⁺ even when nicotinamide is administered. In this respect, sublingual administration of NMN allows direct uptake of NMN into the blood, which is thought to enable efficient synthesis of NAD⁺. That is, sublingual administration of NMN may increase NAD in the body more effectively than oral administration of NAD⁺ precursors (such as nicotinamide) in the elderly.

In order to confirm whether the blood concentration of NMN has increased in a subject or whether NAD in the body could be increased, the NMN concentration in the blood of the subject or the NAD concentration in the body of the subject may be measured after the composition is administered to the subject. The measurement can be performed, for example, for the blood concentration of NMN, by conducting a blood collection within 120 minutes, preferably within 60 minutes, more preferably within 30 minutes after administration of the composition and measuring the NMN concentration in the collected blood using conventional methods.

### [Composition]

### (Form)

The composition of the invention can be a composition that is a food or pharmaceutical. The food and pharmaceutical are not limited to those for humans, and may be those for animals other than human, unless especially noted. The food may be a common food, functional food, or nutritional composition, or a therapeutic diet (diet for the purpose of therapy, which is prepared in accordance with a menu prepared by a dietitian or the like according to a dietary prescription issued by a physician), dietetic food, ingredient-modified food, care food, or therapy-supporting food, unless otherwise stated. The food is not limited to a solid food, but it may be a food in the form of liquid, for example, drink, drinkable preparation, liquid food, or soup, unless otherwise stated. Functional food refers to a food that can give a predetermined functionality to a living body, and includes health foods at large, such as foods for specified health uses (abbreviated as “Tokuho” in Japanese, including conditional foods for specified health use), foods with function claims, foods with health claims including foods with nutrient function claims, foods for special dietary uses, nutritional supplement foods, health supplement foods, supplements, and cosmetic foods (for example, anti-aging agents). For the present invention, the “functional foods” include health foods to which the health claim based on the food standards of CODEX (JOINT FAO/WHO FOOD STANDARDS PROGRAMME CODEX ALIMENTARIUS COMMISSION) is applied.

### (NMN Content, Dose and Usage)

The amount of NMN comprised in the composition of the present invention may be such an amount that the desired effect is exerted. The dose of the composition of the present invention may be appropriately set with taking various factors such as age, weight, symptoms, etc. of the subject into consideration. For example, each unit (one tablet, one agent, dose for one time etc.) can comprise 5 mg or more of NMN, may comprise 10 mg or more, preferably comprises 100 mg or more, more preferably 130 mg or more, more preferably 150 mg or more of NMN, or may comprise 200 mg or more of NMN. The maximum content is not particularly limited, but in any case, it can be 5000 mg or less, or may be 2500 mg or less, or it is preferably 2000 mg or less, more preferably 1000 mg or less, further preferably 750 mg or less.

The amount or concentration of NMN in the composition of the present invention is indicated as the amount or concentration as NMN, unless otherwise stated. That is, when a salt of NMN that is acceptable as food or drug or NAD analogue is used as the active ingredient, such a salt or analogue can be used in an equimolar amount or equimolar concentration of the amount or mole indicated as those of NMN. This conversion can be easily performed by those skilled in the art.

The content of NMN in the composition of the present invention may be designed with taking the daily dose of NMN into account. The daily dose of NMN can be, for example, 5 mg or more, may be 10 mg or more, or is preferably 100 mg or more, more preferably 130 mg or more, further preferably 200 mg or more.

The maximum amount is not particularly limited, but in any case, it can be 5000 mg or less, may be 2500 mg or less, or is preferably 1000 mg or less, more preferably 500 mg or less, further preferably 300 mg or less.

Such a daily dose may be divided into, for example, three parts so that such parts are suitable for three times of administration per day.

The composition may be administered at any point in the day. In view of the fact that NAD concentration in the body is considered to fluctuate during the day and increase during the activity period, in one embodiment, it is considered to be preferred that the composition is administered in a larger amount in the morning, the beginning of the activity period.

The composition of the present invention can be repeatedly administered to a subject and can be continuously administered to a subject for a long period of time. Although the period is not limited, it is preferable to continuously administer for a relatively long period of time, e.g., one week or longer, two weeks or longer, one month or longer, three months or longer, six months or longer, or one year or longer, so that the effect is fully recognized. Since the active ingredient, NMN, is a substance that has been ingested from old days, the composition is particularly suitable for long-term administration.

### (Other Ingredients and Additives)

In one embodiment, the composition of the present invention may further comprise additives acceptable for use in foods or pharmaceuticals. Examples of such additives include inert carriers (solid or liquid carriers), excipients, surfactants, binders, disintegrants, lubricants, dissolution aids, suspending agents, coating agents, colorants, preservatives, buffers, pH adjusters, emulsifiers, stabilizers, sweeteners, antioxidants, flavors, acidulants, and natural products.

In one embodiment, the composition may comprise other active ingredients or nutritional ingredients acceptable for use in foods or pharmaceuticals, in addition to NMN. Examples of such ingredients include sirtuin-activating ingredients (e.g., resveratrol), quercetin, astaxanthin, chlorella, coenzyme Q10, vitamins (e.g., vitamin A, vitamin B1, vitamin B2, vitamin B6, vitamin B12, vitamin C, vitamin D, vitamin E, vitamin K, biotin, folic acid, pantothenic acid, nicotinic acid etc.), amino acids (e.g., lysine, arginine, glycine, alanine, glutamic acid, leucine, isoleucine, valine etc.), carbohydrates (glucose, sucrose, fructose, maltose, trehalose, erythritol, maltitol, palatinose, xylitol, dextrin etc.), electrolytes (e.g., sodium, potassium, calcium, magnesium etc.), minerals (e.g., copper, zinc, iron, cobalt, manganese etc.), antibiotics, dietary fibers, lactic acid bacteria (live and dead bacteria), proteins, lipids, and so forth.

In one embodiment, the composition does not comprise any water-soluble polymer, does not comprise any combination of a water-soluble polymer and surfactant, or does not comprise any combination of a water-soluble polymer, surfactant, and fatty acid. With respect to surfactant and fatty acid, the composition can be made not to comprise a surfactant and fatty acid in such a combination that the HLB values thereof range from about 6 to 15. In another embodiment, the composition does not comprise pullulan, does not comprise any combination of pullulan and glycerin, does not comprise any combination of pullulan, glycerin, and Tween 80, or does not comprise any combination of pullulan, glycerin, Tween 80, and oleic acid.

### (Others)

The composition of the present invention can be used in conjunction with ingestion of other ingredients, ingestion of health foods and supplements, exercise, and normal diet. The composition of the present invention may also be used in combination with activities and exercises that suppress aging. Examples of such activities and exercises that suppress aging include daily activities such as housework, yard work, and walking for commuting to work or shopping, work with a high level of physical activity, hobby and leisure activities, exercise, sports, and so forth.

The composition of the present invention can be manufactured by those skilled in the art using existing facilities and other equipments as appropriate. In the manufacture of the composition, the stage of adding NMN is not particularly limited as long as the properties of NMN are not significantly impaired.

Products containing the composition of the present invention can have an indication of the function and purpose of use (intended use) thereof, and can also have an indication of a recommendation for administration or ingestion thereof to or by a specific target. The indication may be a direct or indirect indication. Examples of the direct indication include descriptions on tangible articles such as the product itself, package, container, label, and tag, and examples of the indirect indication includes advertising and campaign activities using such places or means as web site, shop, pamphlet, exhibition, seminar such as media seminar, book, newspaper, magazine, television, radio, postal matter, E-mail, and sound. Examples of the function and purpose of use (intended use) to be indicated include “for those who want to stay young forever”, “for those who are trying to improve age-related problems”, “for those who want to spend each day young and beautiful”, “for those who are concerned about dry mouth due to aging”, “for those who want to improve oral environment”, “for middle-aged or elderly persons”, “for elderly persons”, and so forth.

The present invention will be further specifically explained with reference to the following examples.

### EXAMPLES

### [Test 1]

There is sublingual absorption as a route of absorption of pharmaceuticals and so forth, and it is known that substances are absorbed directly into the blood by sublingual absorption. That is, if NMN is administered sublingually, NMN is absorbed directly into the blood, and more NMN may be delivered to peripheral tissues through the body circulation. Therefore, it was verified by the following test whether sublingual administration of NMN increases the blood concentration of NMN compared with oral administration.

### Methods

### 1) Medication

### (1) Oral Administration

Tablets were used for oral administration. The content of NMN was 500 mg.

### (2) Sublingual Administration

Powder was used for sublingual administration. The content of NMN was 250 mg.

### 2) Test Subject

One adult male with no abnormal health conditions participated in the test. The test was conducted over two separate days. On both test days, the test was conducted for the subject under the condition that he did not eat breakfast.

### 3) Administration of NMN and Blood Collection

### (1) Oral Administration

A blood sample was taken from the fingertip for a pre-value before administration of NMN. Then, one tablet containing 500 mg of NMN was administered with water, and blood was collected from the fingertip 5, 10, 15, 30, and 60 minutes after the administration. Plasma was separated from the collected blood samples, then diluted 10-fold with 80% methanol, and cryopreserved at −80° C. until measurement.

### (2) Sublingual Administration

A blood sample was taken from the fingertip for a pre-value before administration of NMN. Then, 250 mg of NMN (powder) was placed under the tongue, so that the powder was dissolved with saliva, and the test subject was maintained in that state. After 1 minute, the test subject spit out all NMN from the mouth, and blood was collected from the fingertip 5, 10, 15, and 30 minutes after the NMN was placed under the tongue. Plasma was separated from the collected blood samples, then diluted 10-fold with 80% methanol, and cryopreserved at −80° C. until measurement.

### 4) Determination of NMN Concentration by LC-MS/MS

NMN concentration was determined by LC-MS/MS. Details of the measurements are described below.

The stored methanol-treated blood was thawed, and the supernatant obtained by centrifugation at 4° C. and 15,000 rpm for 10 minutes was evaporated to dryness in a centrifugal evaporator. The dried solid was dissolved in a citric acid solution, the solution was filtered through a 0.22-μm filter, and the filtrate was used as the measurement sample.

The analytical conditions for LC-MS/MS were as follows.
Equipment: Xevo TQ-XS (Waters Corp.)
Column: ACQUITY Premier HSS T3 Column (particle size 1.8 μm, inner diameter 2.1 mm×length 150 mm, Waters Corp.)

### Mobile Phase:

Liquid A: 5 mM Ammonium formate aqueous solution
Liquid B: Acetonitrile

### Gradient Condition of Mobile Phase

From the start of analysis to 3.0 minutes: 0 to 10% of Liquid B
From 3.0 to 3.5 minutes: 10% of Liquid B
From 3.5 to 4.6 minutes: 10 to 90% of Liquid B
From 4.6 to 8.0 minutes: 90% of Liquid B
From 8.0 to 8.1 minutes: 90 to 0% of Liquid B
From 8.1 to 11.5 minutes: 0% of Liquid B
Column temperature: 45° C.
Flow rate: 0.3 mL/minute
Injection volume: 2 μL
Ionization: ESI positive mode
NMN: precursor ion m/z 335, product ion m/z 123

### [Results]

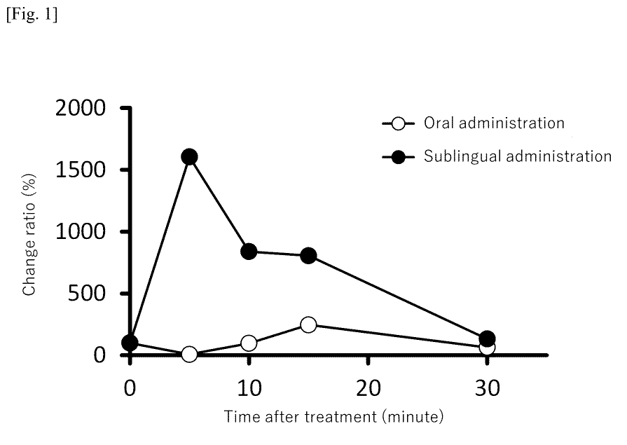

After the oral administration, blood NMN concentration virtually unchanged compared with that observed before NMN ingestion (0 minute) at all the other blood collection points. In contrast, after the sublingual administration, blood NMN concentration increased up to approximately 15-fold compared with that observed before NMN administration (FIG. 1).

### DISCUSSION

The reason why the NMN concentration in the blood hardly changed after oral administration of NMN was thought to be because it was metabolized by the intestinal bacteria and the liver. It was considered that the sublingual administration increased NMN concentration because NMN was rapidly absorbed into the blood without being metabolized.

### [Test 2]

### Methods

### 1) Medication

NMN tablets (each containing 250 mg NMN) were used.

### 2) Test Subject

Two adult males with no abnormal health conditions participated in the test. The test was conducted over two separate days. On both test days, the test was conducted for the subjects under the condition that they did not eat breakfast.

### 3) Administration of NMN and Blood Collection

### (1) Oral Administration

A blood sample was collected from the fingertip for a pre-value before administration of NMN. Then, one tablet of NMN was administered with water, and blood was collected from the fingertip 5, 15, and 30 minutes after the administration. The collected blood samples as whole blood were diluted 10-fold with 80% methanol, and cryopreserved at −80° C. until measurement.

### (2) Oral Mucosa Application

A blood sample was collected from the fingertip for a pre-value before administration of NMN. Then, one tablet of NMN was placed in the mouth, licked, dissolved thereby, and ingested. Blood samples were collected from the fingertips 5, 15, and 30 minutes after the NMN tablet was placed in the mouth. The collected blood samples as whole blood were diluted 10-fold with 80% methanol and cryopreserved at −80° C. until measurement.

### 4) Determination of NMN Concentration by LC-MS/MS

NMN concentration was determined by LC-MS/MS. Details of the measurements are described below.

The stored methanol-treated blood was thawed, and the supernatant obtained by centrifugation at 4° C. and 15,000 rpm for 10 minutes was evaporated to dryness in a centrifugal evaporator. The dried solid was dissolved in ammonium acetate, the solution was filtered through a 0.22-μm filter, and the filtrate was subjected to LC/MS/MS as the measurement sample.

The analytical conditions for LC-MS/MS were as follows.

### Equipment: AB SCIEX QTRAP 4500 LC/MS/MS System (AB SCIEX)

Column: Hypercarb Column (particle size 3 μm, inner diameter 2.1 mm×length 100 mm, Thermo Fischer Scientific)

### Mobile Phase:

Liquid A: 7.5 mM ammonium acetate aqueous solution containing 0.05 (v/v) % ammonium hydroxide
Liquid B: Acetonitrile containing 0.05 (v/v) % ammonium hydroxide

### Gradient Condition of Mobile Phase

From the start of analysis to 1.8 minutes: 5% of Liquid B
From 1.8 to 14 minutes: 5 to 54% of Liquid B
From 14 to 14.1 minutes: 54 to 90% of Liquid B
From 14.1 to 17.1 minutes: 90% of Liquid B
From 17.1 to 17.2 minutes: 90 to 5% of Liquid B
From 17.2 to 32.2 minutes: 5% of Liquid B
Column temperature: 60° C.
Flow rate: 0.2 mL/minute
Injection volume: 3 μL
Ionization: ESI positive mode
NMN: precursor ion m/z 335, product ion m/z 123

### [Results]

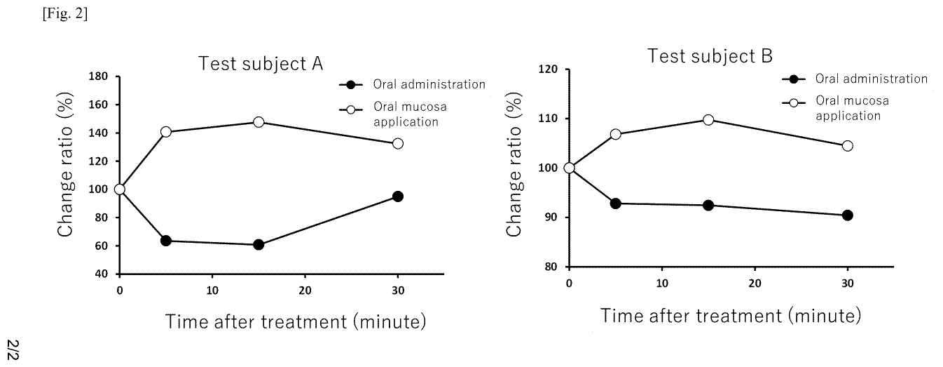

After the oral administration, blood NMN concentration virtually unchanged or decreased compared with that observed before NMN administration (0 minute) at all the other blood collection points. In contrast, after the sublingual administration, blood NMN concentration increased by up to about 15% compared with that observed before NMN administration in both test subjects (FIG. 2).

### DISCUSSION

As in Test 1, the reason why the NMN concentration in the blood hardly changed after oral administration of NMN was thought to be because it was metabolized by the intestinal bacteria and the liver. It was considered that the sublingual administration increased NMN concentration because NMN was rapidly absorbed into the blood without being metabolized. In Test 2, since NMN tablets of the same composition were used, there were not differences in dosage form and dose of NMN, and only the administration route of NMN was the evaluation object. That is, it was found that sublingual administration, which increased the blood concentration of NMN in Test 2, is an administration method that provides more efficient NMN absorption compared with oral administration, which provided no change in the blood concentration.

### CONCLUSION

It was clarified that, as a means for administering NMN, sublingual administration increases the blood concentration of NMN compared with oral administration. The present invention provides an effective means for maximizing the anti-aging effect of NMN.

### Production Examples

### 1) Sublingual Tablet

NMN was mixed with lactose hydrate, cellulose, cyclodextrin, cornstarch, povidone, and Mg stearate, and tablets containing 500 mg of NMN per tablet were produced according to the method for manufacturing oral tablets described in the Japanese Pharmacopoeia.

### 2) Gummi

Gummies containing 250 mg of NMN per gummi were produced in a conventional manner using 30 g of sugar, 50 g of starch syrup, 7 g of gelatin, 5 g of fruit juice concentrate, 1 g of acidulant, and 0.2 g of flavoring as a basic composition.

### 3) Buccal Tablet

Buccal tablets containing 250 mg of NMN per tablet were produced in a conventional manner using D-mannitol, sodium hydrogencarbonate, anhydrous citric acid, dried sodium carbonate, starch sodium glycolate, and magnesium stearate as additives.

### 4) Film Agent

A dispersion solution prepared by uniformly dispersing NMN in a solution dissolving a base component was coated on a plastic film, and the solvent was evaporated to dryness to form a film by cross-linking of the base component. The resulting films were stacked to form a film having an integrated multiple layer structure. The film was cut into a specified size to produce a film agent for intraoral mucosal application containing 200 mg of NMN per agent.

### 5) Tablet

Tablets containing 250 mg of NMN per tablet were produced in a conventional manner using 97 g of saccharides (mainly white sugar), 1.5 g of acidulant, and 0.5 g of flavoring as a basic composition.

### 6) Gum

Gums containing 250 mg of NMN per gum piece was produced in a conventional manner using 30 g of gum base, 63 g of sugar alcohols (maltitol and xylitol), 3 g of flavoring, and 2 g of softener as a basic composition.

### 7) Candy

Candies containing 250 mg NMN per candy were produced in a conventional manner using 43 g of sugar, 45 g of starch syrup, 1 g of acidulant, and 0.2 g of flavoring as a basic composition.

1.-8. (canceled)

9. A method for increasing maximum blood concentration of nicotinamide mononucleotide (NMN) in a subject, comprising:
administering a powder containing NMN to the subject sublingually.

10. The method according to claim 9, wherein the maximum blood concentration of NMN in the subject achieves five times or more than blood concentration of NMN before administration.

11. The method according to claim 9, wherein the subject is selected from elderly, infants and young children.

12. The method according to claim 9, wherein the subject needs anti-aging treatment.

13. The method according to claim 9, wherein the subject needs improvement of oral environment.

14. The method according to claim 9, wherein the powder is selected from a food composition and a pharmaceutical composition.
